(19) United States
(12) Patent Application Publication
(10) Pub. No.: US 2006/0240101 A1
(43) Pub. Date: Oct. 26, 2006

(54) Orally disintegrating pharmaceutical tablet formulations of olanzapine

(76) Inventors: Shubha Chungi, Sharon, MA (US); Claude Richard Barnes, Quakertown, PA (US); Steven M. Lonesky, Schwenksville, PA (US)

Correspondence Address: COOPER & DUNHAM, LLP, 1185 AVENUE OF THE AMERICAS, NEW YORK, NY, 10036, US

(21) Appl. No.: 11/402,416

(22) Filed: Apr. 11, 2006

Related U.S. Application Data
(60) Provisional application No. 60/674,077, filed on Apr. 22, 2005.

(51) Int. Cl. A61K 31/551; A61K 9/20

(52) U.S. Cl. 424/464; 514/220

(57) ABSTRACT

This invention provides orally disintegrating pharmaceutical tablet formulations comprising per tablet the following ingredients in the following percentages by weight: olanzapine as an active ingredient in an amount from about 2.5% to about 10%; mannitol in an amount from about 75% to about 95%; a disintegrating agent in an amount from about 1.0% to about 10%; and one or more excipient in a total amount of about 0.1% to about 10%. This invention also provides a method of treating a patient in need of treatment with olanzapine which comprises administering to the patient a therapeutically effective dose of the above pharmaceutical tablet formulations. Finally, this invention provides a method of producing the above pharmaceutical tablet formulation which comprises combining the ingredients in the appropriate relative percentages by weight.

This application claims the benefit of U.S. Provisional Application No. 60/674,077, filed Apr. 22, 2005, the contents of which are incorporated hereby by reference into the subject application.

Throughout this application, various publications are referenced by full citations. The disclosures of these publications in their entireties are hereby incorporated by reference into this application in order to more fully describe the state of the art as known to those skilled therein.

### FIELD OF THE INVENTION

The present invention relates to orally disintegrating pharmaceutical tablet formulations of 2-methyl-4-(4-methyl-1-piperanzinyl)-10H-thieno[2,3-b][1,5] benzodiazepine, hereinafter referred to as olanzapine, useful in treating psychotic disorders.

### BACKGROUND OF THE INVENTION

Olanzapine is a psychotropic agent that is useful in treating psychotic disorders such as schizophrenia, acute mania in bipolar disorder, and agitation associated with schizophrenia and bipolar disorder. Its molecular formula, molecular weight, and chemical structure, as well as its pharmacological characterization as a selective monoaminergic antagonist that binds with serotonin 5HT_{2A/2C} receptors, dopamine D₁₋₄ receptors, muscarinic M₁₋₅, histamine H₁ receptors, adrenergic α₁ receptors, GABA_{A}, BZD, and β adrenergic receptors have been described in U.S. Pat. No. 5,229,382 and in the 2005 Physician Desk Reference label information for Zyprexa® (olanzapine).

Certain tablet formulations of olanzanpine, are known, as described in U.S. Pat. Nos. 5,229,382, 5,919,485, and 6,190,698, and U.S. Patent Application Publication No. 2001/0018071 A1. In the above patents and patent application, a number of specific tablet formulations of olanzapine are disclosed. In particular, U.S. Pat. No. 5,919,485 discloses the following formulations for one (1) tablet containing 1 mg, 2.5 mg, 5 mg, 7.5 mg, and 10 mg of olanzapine:

| | | | Olanzapine | | | | Olanzapine | | Olanzapine | |
|---|---|---|---|---|---|---|---|---|---|---|
| | Olanzapine | | 2.5 mg | | Olanzapine | | 7.5 mg | | 10 mg | |
| | 1 mg Tablet | | Tablet | | 5 mg Tablet | | Tablet | | Tablet | |
| Olanzapine | 1 | mg | 2.5 | mg | 5 | mg | 7.5 | mg | 10 | mg |
| Lactose | 67.43 | mg | 102.15 | mg | 156 | mg | 234 | mg | 312 | mg |
| Hydroxypropyl | 3.4 | mg | 5.2 | mg | 8 | mg | 12 | mg | 16 | mg |
| Cellulose | | | | | | | | | | |
| Crospovidone | 4.25 | mg | 5.5 | mg | 10 | mg | 15 | mg | 20 | mg |
| Microcrystalline | 8.5 | mg | 13 | mg | 20 | mg | 30 | mg | 40 | mg |
| Cellulose | | | | | | | | | | |
| Magnesium | 0.42 | mg | 0.65 | mg | 1 | mg | 1.5 | mg | 2 | mg |
| Stearate | | | | | | | | | | |
| Hydroxypropyl | 1.7 | mg | 2.6 | mg | 4 | mg | 6 | mg | 8 | mg |
| Methylcellulose | | | | | | | | | | |
| Color Mixture | 3.47 | mg | 5.3 | mg | 8.16 | mg | 12.24 | mg | 16.32 | mg |
| White | | | | | | | | | | |

| | | | | | |
|---|---|---|---|---|---|
| Carnauba Wax | Trace | Trace | Trace | Trace | Trace |
| Edible Blue Ink | Trace | Trace | Trace | Trace | Trace |

These tablet formulations are prepared using a method involving a high shear aqueous wet granulation with fluid bed drying.

Currently, Zyprexa Zydis®, an orally disintegrating tablet formulation of olanzapine marketed by Eli Lilly and Company, is available to treat schizophrenia, bipolar disorder, and agitation associated with schizophrenia and bipolar I mania. According to the 2005 Physician Desk Reference label information for Zyprexa Zydis®, each tablet contains olanzapine equivalent to 5 mg, 10 mg, 15 mg, or 20 mg. It begins disintegrating in the mouth within seconds, allowing its contents to be subsequently swallowed with or without liquid. Zyprexa Zydis® also contains the following ingredients: gelatin, mannitol, aspartame, sodium methyl paraben, and sodium propyl paraben.

In general, absorption of an active ingredient from an oral solid dosage form such as a tablet can be affected by properties of the formulation and its method of production. This is particularly true when the active ingredient, such as olanzapine, is insoluble in water. (2005 Physician Desk Reference label information for Zyprexa®) The dissolution of the active ingredient from an oral solid dosage form in the gastrointestinal tract can be the limiting factor that determines the rate and extent of absorption of active ingredient into the body.

Disintegrating agents can be used to enhance the dissolution rate of a water insoluble active ingredient, such as olanzapine, from an oral solid dosage form. Disintegrating agents are substances or a mixture of substances added to an oral solid dosage form formulation that facilitates the breakup or disintegration of its contents into smaller particles that dissolve more rapidly than in the absence thereof. (Remington: The Science and Practice of Pharmacy, page 862 (Alfonso R. Gennaro ed., 20th ed., 2000)) Materials that serve as disintegrating agents include starches, clays, cellulosics, alginates, gums, and cross-linked polymers. A subgroup of disintegrating agents, known as “super disintegrants,” is known, and is generally used at a low level in solid dosages forms, typically 2% to 4%. Examples of super disintegrants are croscarmellose, crospovidone, and sodium starch glycolate.

In addition, methods for producing fast dissolving tablets are known, as described in U.S. Pat. Nos. 5,501,861, 5,837,285, 6,036,974, and 6,316,026. U.S. Pat. No. 5,501,861 discloses methods for producing fast dissolving tablets by compression molding in a semi-dry state. U.S. Pat. No. 5,837,285 discloses a drug-containing fast soluble tablet which has a pharmaceutical additive rapidly soluble in water as a tablet base component, and is produced using a kneaded mixture of a drug and a pharmaceutical additive rapidly soluble in water that is subjected to compressive shaping while in a wet state. U.S. Pat. No. 5,837,285 also discloses that using mannitol alone results in considerably increased oral cavity dissolution time as well as increased tensile strength when the compressive shaping pressure exceeds 300 kg.

Furthermore, U.S. Pat. No. 6,036,974 discloses methods and apparatuses for preparing molded tablets containing water-soluble or fat-soluble medicines that disintegrate and has a resistance to wear and tear. Specifically, U.S. Pat. No. 6,036,974 discloses mixing active ingredients with an excipient and then kneading together with a binder and solvent into a wetted paste. The top and bottom surfaces of the paste within the mold are coated with powder such as a lubricant to avoid possible sticking of the paste to the apparatus during a subsequent compression step and tablet removal from the mold. U.S. Pat. No. 6,316,026 discloses methods and apparatuses for preparing quick disintegration tablets that have a sufficiently high porosity.

### SUMMARY OF THE INVENTION

This invention provides an orally disintegrating pharmaceutical tablet formulation comprising per tablet the following ingredients in the following percentages by weight: olanzapine as an active ingredient in an amount from about 2.5% to about 10%; mannitol in an amount from about 75% to about 95%; a disintegrating agent in an amount from about 1.0% to about 10%; and one or more excipient in a total amount of about 0.1% to about 10%.

This invention also provides an orally disintegrating pharmaceutical tablet formulation comprising per tablet the following ingredients in the following percentages by weight: olanzapine as an active ingredient in an amount from about 2.5% to about 10%; mannitol in an amount from about 86.5% to about 94%; a sodium starch glycolate in an amount of about 3% wherein the sodium starch glycolate is a cross-linked, low-substituted carboxymethyl ether of poly-α-glucopyranose obtained from potato starch, and has a median particle size in the range from about 35 μm to about 55 μm; and an excipient in an amount of about 0.5%.

The invention also provides a method of treating a patient in need of treatment with olanzapine which comprises administering to the patient a therapeutically effective dose of any of the above pharmaceutical tablet formulations.

### DETAILED DESCRIPTION OF THE INVENTION

This invention provides an orally disintegrating pharmaceutical tablet formulation comprising per tablet the following ingredients in the following percentages by weight: olanzapine as an active ingredient in an amount from about 2.5% to about 10%; mannitol in an amount from about 75% to about 95%; a disintegrating agent in an amount from about 1.0% to about 10%; and one or more excipient in a total amount of about 0.1% to about 10%.

As used herein, a “disintegrating agent” is a substance or a mixture of substances added to a pharmaceutical tablet formulation that facilitates its breakup or disintegration of the tablet contents into smaller particles that dissolve more rapidly than in the absence thereof. Examples of disintegrating agent include starches, clays, celluloses, algins, gums, and cross-linked polymers. Disintegrating agents also include croscarmellose, crospovidone, and sodium starch glycolate.

A preferred disintegrating agent in the present invention is a sodium starch glycolate, such as Explotab® from JRS Pharma LP, Patterson, New Jersey, which is a cross-linked, low-substituted carboxymethyl ether of poly-α-glycopyranose obtained from potato starch, which has a medium particle size of about 200 μm. Such a sodium starch glycolate disintegrating agent provides considerable disintegration and dissolution efficiency when incorporated in tablet formulations prepared by direct compression or by wet or dry granulation techniques. The advantages of such sodium starch glycolate disintegrating agents are ability to maintain its swollen granules intact, no secondary binding, uniform particle-size range, high bulk density, low use levels, long shelf-life stability, and compatibility in the broadest spectrum of formulations.

Tablets in accordance with the invention may contain a number of suitable inert materials or “excipients.” As used herein, an “excipient” is one suitable for use with humans and/or animals without undue adverse side effects, such as toxicity, irritation, and allergic response, commensurate with a reasonable benefit/risk ratio. Excipients that may be used to help impart satisfactory processing and compression characteristics to the tablet formulation include diluents (e.g., dicalcium phosphate, calcium sulfate, lactose, cellulose, kaolin, sodium chloride, dry starch, powdered sugar, mannitol, lactose, sorbitol, sucrose, inositol, bentonite, and microcrystalline cellulose), binders (e.g., starch, gelatin, natural sugars such as sucrose, glucose, dextrose, molasses, and lactose, natural and synthetic gums such as sodium alginate, carboxymethylcellulose, polyethylene glycol, waxes, water, and alcohol, such as ethyl alcohol) glidants, and lubricants (e.g., sodium oleate, sodium stearate, magnesium stearate, sodium benzoate, sodium acetate, sodium chloride, and the like). Excipients that may be used to help give additional desirable physical characteristics to the tablet formulation include coloring agents (e.g., water-soluble Food, Drug and Cosmetic Act (FD&C) colors, such as FD&C Blue No. 2 (12%-14%), Color Yellow #6 FD&C Alum Lake, and Color Red #40 FD&C Lake 35-42%), and flavoring agents (e.g., mannitol, lactose, and artificial sweetening agents such as Prosweet® N&A FL Powder from Virginia Dare Extract Co., Inc., Brooklyn, N.Y.).

In one embodiment, the orally disintegrating pharmaceutical tablet formulation comprises per tablet the following ingredients in the following percentages by weight: olanzapine as an active ingredient in an amount from about 2.5% to about 10%; mannitol in an amount from about 86.5% to about 94%; a sodium starch glycolate in an amount of about 3% wherein the sodium starch glycolate is a cross-linked, low-substituted carboxymethyl ether of poly-α-glucopyranose obtained from potato starch, and has a median particle size in the range from about 35 μm to about 55 μm; and an excipient in an amount of about 0.5%.

In a presently preferred embodiment of the invention, the mannitol comprises a mixture of a first mannitol that has a mean particle size of about 35 μm and is water soluble at 20° C. at approximately 250 g/l; and a second mannitol that has a mean particle size of about 200 μm, a porous crystalline particle structure, and is water soluble at 20° C. at approximately 170 g/l.

As used herein, a preferred “first mannitol” is a mannitol that has a mean particle size of about 35 μm. and is water soluble at 20° C. at approximately 250 g/l available under the trademark Pearlitol® 50C (formerly known as Mannitol 35, NF) from Roquette Frères, Lestrem, France.

As used herein, a preferred “second mannitol” is a mannitol that has a mean particle size of about 200 μm, a porous crystalline particle structure, and is water soluble at 20° C. at approximately 170 g/l available under the trademark Pearlitol® 200 SD from Roquette Frères, Lestrem, France.

This invention further provides the above pharmaceutical tablet formulations, wherein in the mixture the first mannitol is present in an amount from about 79% to about 86% and the second mannitol is present in an amount from about 7.5% to about 8.25%.

The invention further provides the above pharmaceutical tablet formulations, wherein the excipient comprises a flavoring agent alone or together with a coloring agent.

As stated above, coloring agents, such as water-soluble Food, Drug and Cosmetic Act (FD&C) colors, such as FD&C Blue No. 2 (12%-14%), Color Yellow #6 FD&C Alum Lake, and Color Red #40 FD&C Lake 35-42%), and/or flavoring agents, such as mannitol and artificial sweetening agents, including Prosweet® N&A FL Powder, may be used to help give additional desirable physical characteristics to the tablet formulation. (Remington: The Science and Practice of Pharmacy, pages 862 and 863 (Alfonso R. Gennaro ed., 20th ed., 2000)) Presently, preferred coloring agents are FD&C Blue No. 2 (12%-14%), Color Yellow #6 FD&C Alum Lake, and Color Red #40 FD&C Lake 35-42%. A presently preferred flavoring agent is Prosweet® N&A FL Powder.

In one embodiment of this invention, the pharmaceutical tablet formulation comprises per tablet the following ingredients in the following percentages by weight: olanzapine in an amount of about 2.5%; the first mannitol in an amount of about 85.55%; the second mannitol in an amount of about 8.25%; the sodium starch glycolate in an amount of about 3%; and excipient in an amount of about 0.7%.

In another embodiment of this invention, the pharmaceutical tablet formulation comprises per tablet the following ingredients in the following percentages by weight: olanzapine in an amount of about 5%; the first mannitol in an amount of about 83.4%; the second mannitol in an amount of about 8%; the sodium starch glycolate in an amount of about 3%; and excipient in an amount of about 0.6%.

In yet another embodiment of this invention, the pharmaceutical tablet formulation comprises per tablet the following ingredients in the following percentages by weight: olanzapine in an amount of about 7.5%; the first mannitol in an amount of about 81.15%; the second mannitol in an amount of about 7.75%; the sodium starch glycolate in an amount of about 3%; and excipient in an amount of about 0.6%.

In still another embodiment of this invention, the pharmaceutical tablet formulation comprises per tablet the following ingredients in the following percentages by weight: olanzapine in an amount of about 10%; the first mannitol in an amount of about 79%; the second mannitol in an amount of about 7.5%; the sodium starch glycolate in an amount of about 3%; and excipient in an amount of about 0.5%.

The invention also provides a method of treating a patient in need of treatment with olanzapine which comprises administering to the patient a therapeutically effective dose of any of the above described pharmaceutical tablet formulations.

“A patient in need of treatment with olanzapine” includes but is not limited to a patient suffering from a central nervous system disorder, particularly a psychotic disorder, such as schizophrenia, bipolar disorder, or agitation associated with schizophrenia and bipolar I mania, or a patient in need of maintenance treatment in bipolar disorder.

A “therapeutically effective dose” of any of the above pharmaceutical tablet formulations is a number and frequency of administration of the tablets of this invention which provides to a typical adult patient a dose of about 0.25 mg olanzapine/day to about 50 mg olanzapine/day, preferably from about 1 mg olanzapine/day to about 30 mg olanzapine/day, and most preferably from about 5 mg olanzapine/day to about 20 mg olanzapine/day. Typical unit dosage forms are 5 mg olanzapine/day, 10 mg olanzapine/day, 15 mg olanzapine/day, or 20 mg olanzapine/day.

The “administrating to the patient” is typically effected orally once a day or multiple times during a day. The above pharmaceutical tablet formulations can be administered alone or in combination with concomitant lithium or valproate treatment.

The invention also provides a method of producing any of the above pharmaceutical tablet formulations which comprises combining the ingredients thereof in the appropriate relative percentages by weight to produce the tablets using conventional tableting methods as described more fully herein. Tablets in accordance with the invention have the characteristic that they can be easily and readily prepared with high batch to batch consistency.

### EXPERIMENTAL DETAILS

### Example 1

### Olanzapine Orally Disintegrating Pharmaceutical Tablet (5 mg/Tablet)

Purified water, USP (0.742 kg) and ethyl alcohol, USP 160 proof, (3.712 kg) are mixed in a stainless steel container for two minutes with a spatula. The mixture of purified water and ethyl alcohol is then tightly closed in the container to prevent further evaporation and set aside for later use. The dry ingredients, i.e., a portion of Mannitol 35, NF (4.403 kg (about 14.83% of the total weight of tablet)), olanzapine (0.742 kg (about 2.5%)), sodium starch glycolate (Explotab®) (0.891 kg (about 3%)), Prosweet® N&A FL Powder (0.148 kg (about 0.5%)), Color Red #40 FD&C Lake 35-42% (0.059 kg (about 0.2%)), and Mannitol, NF (Pearlitol® 200SD) (2.450 kg (about 8.25%)) are passed in order, separately, through a #16 wire stainless steel screen or equivalent, and loaded, in order, into a two cubic foot twin shell V-blender with intensifier bar. The dry ingredients are blended for 5 minutes with the intensifier bar on at 1200 RPM. The remaining Mannitol 35, NF (21.0 kg (about 70.72%)) is added to the dry blended ingredients by passing the remaining Mannitol 35, NF through a #16 wire stainless steel screen or equivalent and loading it into the two cubic foot twin shell V-blender with intensifier bar. The dry ingredients with the remaining Mannitol 35, NF is blended for 10 minutes with the intensifier bar on at 1200 RPM. Using a pump equipped with Tygon® Master Flex Tubing from Saint Gobain Performance Plastics, Akron, Ohio into the intensifier bar port of the twin shell blender, the mixture of purified water and ethyl alcohol made earlier is then added to the blended dry ingredients. The wet mixture is blended in the two cubic foot twin shell V-blender with intensifier bar for 30 seconds with the intensifier bar at 1200 RPM. The wet blended mixture is loaded in a triturate machine (Vector Model 820 Automated Triturate Machine® from Vector Corporation, Marion, Iowa) which forms the wet blended mixture into wet molded tablet forms by a molding pressure of about 6.3 kg/cm² or about 90 psi. The wet molded tablet forms are then dried in a hot air drying oven at 50° F. until the tablet forms exhibit a loss on drying moisture endpoint of 1%-2%.

Table 1 provides the formulation which is used in conjunction with the above detailed process to produce an orally disintegrating pharmaceutical tablet formulation containing 5 mg of olanzapine per tablet:

TABLE 1
Orally Disintegrating Tablet Pharmaceutical
Formulations Containing 5 mg of Olanzapine Per Tablet

5 mg

| INGREDIENT | mg/Tablet | g/Batch |
|---|---|---|
| Olanzapine | 5 | 742 |
| Mannitol 35, NF | 171.1 | 25,403 |
| Mannitol, NF | 16.5 | 2,450 |
| (Pearlitol 200 SD ®) | | |
| Explotab ®, NF | 6 | 891 |
| (Sodium Starch | | |
| Glycolate) | | |
| Prosweet N&A FL | 1 | 148 |
| Powder | | |
| FD&C Blue No. 2 | — | — |
| Lake (12-14%) | | |
| Color Yellow #6 | — | — |
| FD&C Alum Lake | | |
| Color Red #40 | 0.4 | 59 |
| FD&C Lake 35-42% | | |
| Ethyl Alcohol, USP | Non-Residual | 3,712 |
| 190 Proof | | |
| Purified Water, USP | Non-Residual | 742 |
| Total Weight | 200 | 29,693 |
| (Dry Ingredients) | | |

### Example 2

### Olanzapine Orally Disintegrating Pharmaceutical Tablet (10 mg/Tablet)

Purified water, USP (0.742 kg) and ethyl alcohol, USP 160 proof, (3.712 kg) are mixed in a stainless steel container for two minutes with a spatula. The mixture of purified water and ethyl alcohol is then tightly closed in the container to prevent further evaporation and set aside for later use. The dry ingredients, i.e., a portion of Mannitol 35, NF (4.765 kg (about 16.05% of the total weight of tablet)), olanzapine (1.485 kg (about 5%)), sodium starch glycolate (Explotab®) (0.891 kg (about 3%)), Prosweet® N&A FL Powder (0.148 kg (about 0.5%)), Color Yellow #6 FD&C Alum Lake (0.030 kg (about 0.1%)), and Mannitol, NF (Pearlitol® 200 SD) (2.375 kg (about 8%)) are passed in order, separately, through a #16 wire stainless steel screen or equivalent, and loaded, in order, into a two cubic foot twin shell V-blender with intensifier bar. The dry ingredients are blended for 5 minutes with the intensifier bar on at 1200 RPM. The remaining Mannitol 35, NF (20.0 kg (about 67.35%)) is added to the dry blended ingredients by passing the remaining Mannitol 35, NF through a #16 wire stainless steel screen or equivalent and loading it into the two cubic foot twin shell V-blender with intensifier bar. The dry ingredients with the remaining Mannitol 35, NF is blended for 10 minutes with the intensifier bar on at 1200 RPM. Using a pump equipped with Tygon® Master Flex Tubing into the intensifier bar port of the twin shell blender, the mixture of purified water and ethyl alcohol made earlier is then added to the blended dry ingredients. The wet mixture is blended in the two cubic foot twin shell V-blender with intensifier bar for 30 seconds with the intensifier bar at 1200 RPM. The wet blended mixture is loaded in a triturate machine (Vector Model 820 Automated Triturate Machine@) which forms the wet blended mixture into wet molded tablet forms by a molding pressure of about 6.3 kg/cm² or about 90 psi. The wet molded tablet forms are then dried in a hot air drying oven at 50° F. until the tablet forms exhibit a loss on drying moisture endpoint of 1%-2%.

Table 2 provides the formulation which is used in conjunction with the above detailed process to produce an orally disintegrating pharmaceutical tablet formulation containing 10 mg of olanzapine per tablet:

TABLE 2
Orally Disintegrating Pharmaceutical Tablet
Formulations Containing 10 mg of Olanzapine Per Tablet

10 mg

| INGREDIENT | mg/Tablet | g/Batch |
|---|---|---|
| Olanzapine | 10 | 1,485 |
| Mannitol 35, NF | 166.8 | 24,765 |
| Mannitol, NF | 16 | 2,375 |
| (Pearlitol 200 SD ®) | | |
| Explotab ®, NF | 6 | 891 |
| (Sodium Starch | | |
| Glycolate) | | |
| Prosweet N&A FL | 1 | 148 |
| Powder | | |
| FD&C Blue No. 2 | — | — |
| Lake (12-14%) | | |
| Color Yellow #6 | 0.2 | 30 |
| FD&C Alum Lake | | |
| Color Red #40 | — | — |
| FD&C Lake 35-42% | | |
| Ethyl Alcohol, USP | Non-Residual | 3,712 |
| 190 Proof | | |
| Purified Water, USP | Non-Residual | 742 |
| Total Weight | 200 | 29,694 |
| (Dry Ingredients) | | |

### Example 3

### Olanzapine Orally Disintegrating Pharmaceutical Tablet (15 mg/Tablet)

Purified water, USP (0.742 kg) and ethyl alcohol, USP 160 10 proof, (3.712 kg) are mixed in a stainless steel container for two minutes with a spatula. The mixture of purified water and ethyl alcohol is then tightly closed in the container to prevent further evaporation and set aside for later use. The dry ingredients, i.e., a portion of Mannitol 35, NF (4.097 kg (about 13.8% of the total weight of tablet)), olanzapine (2.227 kg (about 7.5%)), sodium starch glycolate (Explotab®) (0.891 kg (about 3%)), Prosweet® N&A FL Powder (0.148 kg (about 0.5%)), FD&C Blue No. 2 Lake (12-14%) (0.030 kg (about 0.1%)), and Mannitol, NF (Pearlitol® 200 SD) (2.301 kg (about 7.75%)) are passed in order, separately, through a #16 wire stainless steel screen or equivalent, and loaded, in order, into a two cubic foot twin shell V-blender with intensifier bar. The dry ingredients are blended for 5 minutes with the intensifier bar on at 1200 RPM. The remaining Mannitol 35, NF (20.0 kg (about 67.35%)) is added to the dry blended ingredients by passing the remaining Mannitol 35, NF through a #16 wire stainless steel screen or equivalent and loading it into the two cubic foot twin shell V-blender with intensifier bar. The dry ingredients with the remaining Mannitol 35, NF is blended for 10 minutes with the intensifier bar on at 1200 RPM. Using a pump equipped with Tygon® Master Flex Tubing into the intensifier bar port of the twin shell blender, the mixture of purified water and ethyl alcohol made earlier is then added to the blended dry ingredients. The wet mixture is blended in the two cubic foot twin shell V-blender with intensifier bar for 30 seconds with the intensifier bar at 1200 RPM. The wet blended mixture is loaded in a triturate machine (Vector Model 820 Automated Triturate Machine®) which forms the wet blended mixture into wet molded tablet forms by a molding pressure of about 6.3 kg/cm² or about 90 psi. The wet molded tablet forms are then dried in a hot air drying oven at 50° F. until the tablet forms exhibit a loss on drying moisture endpoint of 1%-2%.

Table 3 provides the formulation which is used in conjunction with the above detailed process to produce an orally disintegrating pharmaceutical tablet formulation containing 15 mg of olanzapine per tablet:

TABLE 3
Orally Disintegrating Pharmaceutical Tablet
Formulations Containing 15 mg of Olanzapine Per Tablet

15 mg

| INGREDIENT | mg/Tablet | g/Batch |
|---|---|---|
| Olanzapine | 15 | 2,227 |
| Mannitol 35, NF | 162.3 | 24,097 |
| Mannitol, NF | 15.5 | 2,301 |
| (Pearlitol 200 SD ®) | | |
| Explotab ®, NF | 6 | 891 |
| (Sodium Starch | | |
| Glycolate) | | |
| Prosweet N&A FL | 1 | 148 |
| Powder | | |
| FD&C Blue No. 2 | 0.2 | 30 |
| Lake (12-14%) | | |
| Color Yellow #6 | — | — |
| FD&C Alum Lake | | |
| Color Red #40 | — | — |
| FD&C Lake 35-42% | | |
| Ethyl Alcohol, USP | Non- | 3,712 |
| 190 Proof | Residual | |
| Purified Water, USP | Non- | 742 |
| | Residual | |
| Total Weight | 200 | 29,694 |
| (Dry Ingredients) | | |

### Example 4

### Olanzapine Orally Disintegrating Pharmaceutical Tablet (20 mg/Tablet)

Purified water, USP (0.742 kg) and ethyl alcohol, USP 160 proof, (3.712 kg) are mixed in a stainless steel container for two minutes with a spatula. The mixture of purified water and ethyl alcohol is then tightly closed in the container to prevent further evaporation and set aside for later use. The dry ingredients, i.e., a portion of Mannitol 35, NF (4.46 kg (about 15.02% of the total weight of tablet), olanzapine (2.969 kg (about 10%)), sodium starch glycolate (Explotab®) (0.891 kg (about 3%)), Prosweet® N&A FL Powder (0.148 kg (about 0.5%)), and Mannitol, NF (Pearlitol® 200 SD) (2.226 kg (about 7.5%)) are passed in order, separately, through a #16 wire stainless steel screen or equivalent, and loaded, in order, into a two cubic foot twin shell V-blender with intensifier bar. The dry ingredients are blended for 5 minutes with the intensifier bar on at 1200 RPM. The remaining Mannitol 35, NF (19.0 kg (about 63.99%)) is added to the dry blended ingredients by passing the remaining Mannitol 35, NF through a #16 wire stainless steel screen or equivalent and loading it into the two cubic foot twin shell V-blender with intensifier bar. The dry ingredients with the remaining Mannitol 35, NF is blended for 10 minutes with the intensifier bar on at 1200 RPM. Using a pump equipped with Tygon® Master Flex Tubing into the intensifier bar port of the twin shell blender, the mixture of purified water and ethyl alcohol made earlier is then added to the blended dry ingredients. The wet mixture is blended in the two cubic foot twin shell V-blender with intensifier bar for 30 seconds with the intensifier bar at 1200 RPM. The wet blended mixture is loaded in a triturate machine (Vector Model 820 Automated Triturate Machine®) which forms the wet blended mixture into wet molded tablet forms by a molding pressure of about 6.3 kg/cm² or about 90 psi. The wet molded tablet forms are then dried in a hot air drying oven at 50° F. until the tablet forms exhibit a loss on drying moisture endpoint of 1%-2%.

Table 4 provides the formulation which is used in conjunction with the above detailed process to produce an orally disintegrating pharmaceutical tablet formulation containing 20 mg of olanzapine per tablet:

TABLE 4
Orally Disintegrating Pharmaceutical Tablet
Formulations Containing 20 mg of Olanzapine Per Tablet

20 mg

| INGREDIENT | mg/Tablet | g/Batch |
|---|---|---|
| Olanzapine | 20 | 2,969 |
| Mannitol 35, NF | 158 | 23,460 |
| Mannitol, NF | 15 | 2,226 |
| (Pearlitol 200 SD ®) | | |
| Explotab ®, NF | 6 | 891 |
| (Sodium Starch | | |
| Glycolate) | | |
| Prosweet N&A FL | 1 | 148 |
| Powder | | |
| FD&C Blue No. 2 | — | — |
| Lake (12-14%) | | |
| Color Yellow #6 | — | — |
| FD&C Alum Lake | | |
| Color Red #40 | — | — |
| FD&C Lake 35-42 | | |
| Ethyl Alcohol, USP | Non-Residual | 3,712 |
| 190 Proof | | |
| Purified Water, USP | Non-Residual | 742 |
| Total Weight | 200 | 29,694 |
| (Dry Ingredients) | | |

The above tablet formulations of olanzapine have a friability target of less than 2% and a disintegration time of 20 seconds or less.

The stability of each of the 5 mg, 10 mg, 15 mg and 20 mg tablet formulations of olanzapine, is tested at 25° C.±2° C./60% RH±5% RH, over a 24-month period by chemical analysis of the triturates. Additionally or alternatively, stability may be tested at accelerated conditions of 40° C./75% RH for 12 weeks which corresponds to a 24-month shelf life. Stability is verified by an olanzapine HPLC assay and an organic HPLC assay of total impurities. The 5 mg, 10 mg, 15 mg and 20 mg tablet formulations of olanzapine are found to be stable throughout the period of testing.

The above tablet formulations of olanzapine are characterized as having a porosity of about 34%. Porosity (%) is determined as follows:

$$100 \times \frac{\begin{matrix}Volume of Tablet - ( Weight of Tablet ) / \\ ( True Density of Ingredients )\end{matrix}}{Volume of Tablet}$$

wherein the “Volume of Tablet” is 0.1887 mL, the “Weight of Tablet” is 0.204 g, and the “True Density of Ingredients” is 1.636 g/mL.

The above tablet formulations of olanzapine also have a density of between about 1060-1100 mg/ml, and a falling impact strength of about 0.12%. Density maybe determined by pouring a tablet into a graduated cylinder and measuring the volume the tablet occupies divided by the total weight of the sample. Falling impact strength maybe determined by dropping a tablet from a height of 30 cm onto a glass plate and measuring the % degree of destruction.

The above tablet formulations of olanzapine are further characterized as having a hardness (crushing strength) of about 1-6 kp with the average hardness being about 3 kp (1 kp=1 kgf). Hardness is determined by using a standard hardness tester, such as a Dr. Schleuniger® Tablet Hardness Tester from Dr. Schleuniger Pharmatron AG, Solothum, Switzerland.

1. An orally disintegrating pharmaceutical tablet formulation comprising per tablet the following ingredients in the following percentages by weight:
a) olanzapine as an active ingredient in an amount from about 2.5% to about 10%;
b) mannitol in an amount from about 75% to about 95%;
c) a disintegrating agent in an amount from about 1.0% to about 10%; and
d) one or more excipient in a total amount of about 0.1% to about 10%.

2. The pharmaceutical tablet formulation of claim 1 comprising per tablet the following ingredients in the following percentages by weight:
a) olanzapine as an active ingredient in an amount from about 2.5% to about 10%;
b) mannitol in an amount from about 86.5% to about 94%,
c) a sodium starch glycolate in an amount of about 3% wherein the sodium starch glycolate is a cross-linked, low-substituted carboxymethyl ether of poly-α-glucopyranose obtained from potato starch, and has a median particle size in the range from about 35 μm to about 55 μm; and
d) an excipient in an amount of about 0.5%.

3. The pharmaceutical tablet formulation of claim 1, wherein the mannitol comprises a mixture of a first mannitol that has a mean particle size of about 35 {μm and is water soluble at 20° C. at approximately 250 g/l; and a second mannitol that has a mean particle size of about 200 μm, a porous crystalline particle structure, and is water soluble at 20° C. at approximately 170 g/l.

4. The pharmaceutical tablet formulation of claim 3, wherein in the mixture the first mannitol is present in an amount from about 79% to about 86% and the second mannitol is present in an amount from about 7.5% to about 8.25%.

5. The pharmaceutical tablet formulation of claim 1, wherein the excipient comprises a flavoring agent alone or together with a coloring agent.

6. The pharmaceutical tablet formulation of claim 2, comprising per tablet the following ingredients in the following percentages by weight:
a) olanzapine in an amount of about 2.5%;
b) the first mannitol in an amount of about 85.55%;
c) the second mannitol in an amount of about 8.25%;
d) the sodium starch glycolate in an amount of about 3%; and
e) excipient in an amount of about 0.7%.

7. The pharmaceutical tablet formulation of claim 2, comprising per tablet the following ingredients in the following percentages by weight:
a) olanzapine in an amount of about 5%;
b) the first mannitol in an amount of about 83.4%;
c) the second mannitol in an amount of about 8%;
d) the sodium starch glycolate in an amount of about 3%; and
e) excipient in an amount of about 0.6%.

8. The pharmaceutical tablet formulation of claim 2, comprising per tablet the following ingredients in the following percentages by weight:
a) olanzapine in an amount of about 7.5%;
b) the first mannitol in an amount of about 81.15%;
c) the second mannitol in an amount of about 7.75%;
d) the sodium starch glycolate in an amount of about 3%; and
e) excipient in an amount of about 0.6%.

9. The pharmaceutical tablet formulation of claim 2, comprising per tablet the following ingredients in the following percentages by weight:
a) olanzapine in an amount of about 10%;
b) the first mannitol in an amount of about 79%;
c) the second mannitol in an amount of about 7.5%;
d) the sodium starch glycolate in an amount of about 3%; and
e) excipient in an amount of about 0.5%.

10. A method of treating a patient in need of treatment with olanzapine which comprises administering to the patient a therapeutically effective dose of the pharmaceutical tablet formulation of claims 1-9.

11. A method of producing the pharmaceutical tablet formulation of claims 1 which comprises combining the ingredients in the relative percentages by weight recited in such claim.

12. The pharmaceutical tablet formulation of claim 2, wherein the mannitol comprises a mixture of a first mannitol that has a mean particle size of about 35 μm and is water soluble at 20° C. at approximately 250 g/l; and a second mannitol that has a mean particle size of about 200 μm, a porous crystalline particle structure, and is water soluble at 20° C. at approximately 170 g/l.

13. The pharmaceutical tablet formulation of claim 2, wherein the excipient comprises a flavoring agent alone or together with a coloring agent.

14. The pharmaceutical tablet formulation of claim 3, wherein the excipient comprises a flavoring agent alone or together with a coloring agent.

15. The pharmaceutical tablet formulation of claim 4, wherein the excipient comprises a flavoring agent alone or together with a coloring agent.

16. The pharmaceutical tablet formulation of claim 12, wherein the excipient comprises a flavoring agent alone or together with a coloring agent.

17. A method of treating a patient in need of treatment with olanzapine which comprises administering to the patient a therapeutically effective dose of the pharmaceutical tablet formulation of claim 2.

18. A method of treating a patient in need of treatment with olanzapine which comprises administering to the patient a therapeutically effective dose of the pharmaceutical tablet formulation of claim 12.

19. A method of producing the pharmaceutical tablet formulation of claim 2 which comprises combining the ingredients in the relative percentages by weight recited in such claim.

20. A method of producing the pharmaceutical tablet formulation of claim 12 which comprises combining the ingredients in the relative percentages by weight recited in such claim.
